(19) United States
(12) Patent Application Publication
(10) Pub. No.: US 2020/0249217 A1
(43) Pub. Date: Aug. 6, 2020

(54) SYSTEM AND METHOD FOR MONITORING SOIL CONDITIONS BASED ON DATA RECEIVED FROM A SENSOR MOUNTED WITHIN A NON-ROTATING TOOL

(71) Applicant: CNH Industrial America, LLC, New Holland, PA (US)

(72) Inventors: Trevor Stanhope, Palos Hills, IL (US); Jason Fox, Chicago, IL (US)

(21) Appl. No.: 16/268,622

(22) Filed: Feb. 6, 2019

(51) Int. Cl. G01N 33/24; A01B 3/24; A01B 79/00; A01B 15/02; A01B 63/24; A01B 76/00; G01N 21/25

(52) U.S. Cl. CPC G01N 33/246; A01B 3/24; A01B 79/005; A01B 15/02; G01N 2033/243; A01B 76/00; G01N 21/25; G01N 2033/245; A01B 63/24

(57) ABSTRACT

In one aspect, a system for monitoring soil composition within a field may include a non-rotating ground-engaging tool configured to be pulled through soil within the field in a manner that performs an agricultural operation on the field. The non-rotating ground-engaging tool may, in turn, define a cavity therein, with the cavity including an opening. Furthermore, the system may include a sensor positioned within the cavity, with the sensor configured emit an output signal through the opening for reflection off of the soil within the field. The sensor may also be configured to detect the reflected output signal as a return signal, with a parameter of the return signal being indicative of a soil composition of the soil within the field.

### FIELD OF THE INVENTION

The present disclosure generally relates to agricultural machines and, more particularly, to systems and methods for monitoring soil conditions within a field across which an agricultural machine is moved based on data received from a sensor installed or otherwise mounted within a non-rotating ground-engaging tool of the machine.

### BACKGROUND OF THE INVENTION

It is well known that, to attain the best agricultural performance from a field, a farmer must cultivate the soil, typically through a tillage operation. Modern farmers perform tillage operations by pulling a tillage implement behind an agricultural work vehicle, such as a tractor. Tillage implements typically include a plurality of ground-engaging tools, such as harrow discs, shanks, leveling discs, tines, rolling baskets, and/or the like, which loosen and/or otherwise agitate the soil to prepare the soil for subsequent planting operations.

Upon completion of the tillage operation, it is generally desirable that the field have a certain soil composition, such as particular organic matter, residue, and/or moisture content. However, due to varying conditions across the field and/or other factors, it may be necessary to adjust one or more operating parameters of the tillage implement during the tillage operation to ensure that the field has such soil composition. In this regard, systems and methods for monitoring the soil composition within a field have been developed. However, further improvements to such systems and methods are needed.

Accordingly, an improved system and method for monitoring soil conditions within a field would be welcomed in the technology.

### SUMMARY OF THE INVENTION

Aspects and advantages of the technology will be set forth in part in the following description, or may be obvious from the description, or may be learned through practice of the technology.

In one aspect, the present subject matter is directed to a system for monitoring soil composition within a field. The system may include a non-rotating ground-engaging tool configured to be pulled through soil within the field in a manner that performs an agricultural operation on the field. The non-rotating ground-engaging tool may, in turn, define a cavity therein, with the cavity including an opening. Furthermore, the system may include a sensor positioned within the cavity, with the sensor configured emit an output signal through the opening for reflection off of the soil within the field. The sensor may also be configured to detect the reflected output signal as a return signal, with a parameter of the return signal being indicative of a soil composition of the soil within the field.

In a further aspect, the present subject matter is directed to an agricultural implement. The agricultural implement may include a frame and a non-rotating ground-engaging tool mounted on the frame. The non-rotating ground-engaging tool may, in turn, be configured to be pulled through soil within the field in a manner that performs an agricultural operation on the field as the agricultural implement is moved across the field. The non-rotating ground-engaging tool may, in turn, define a cavity therein, with the cavity including an opening. The agricultural implement may also include a sensor positioned within the cavity, with the sensor configured emit an output signal through the opening for reflection off of the soil within the field. The sensor may also be configured to detect the reflected output signal as a return signal, with a parameter of the return signal is indicative of a soil composition of the soil within the field.

In a further aspect, the present subject matter is directed to a method for monitoring soil composition within a field across which an agricultural machine is being moved. The agricultural machine may include a non-rotating ground-engaging tool configured to be pulled through soil within the field in a manner that performs an agricultural operation on the field. The non-rotating ground-engaging tool may define a cavity therein, with the cavity including an opening. The method may include receiving, with a computing device, data from a sensor positioned within the cavity. The sensor may be configured to emit an output signal through the opening for reflection off of the soil within the field and detect the reflected output signal as a return signal. The method may also include determining, with the computing device, a soil composition of the soil based on the received data. Furthermore, when the determined soil composition of the soil differs from a predetermined range of soil compositions, the method may include initiating, with the computing device, a control action associated with adjusting an operating parameter of the agricultural machine.

These and other features, aspects and advantages of the present technology will become better understood with reference to the following description and appended claims. The accompanying drawings, which are incorporated in and constitute a part of this specification, illustrate embodiments of the technology and, together with the description, serve to explain the principles of the technology.

### BRIEF DESCRIPTION OF THE DRAWINGS

A full and enabling disclosure of the present technology, including the best mode thereof, directed to one of ordinary skill in the art, is set forth in the specification, which makes reference to the appended figures, in which:

FIG. 1 illustrates a perspective view of one embodiment of an agricultural machine in accordance with aspects of the present subject matter:

FIG. 2 illustrates a side view of one embodiment of a non-rotating, ground-engaging tool of an agricultural machine in accordance with aspects of the present subject matter;

FIG. 3. Illustrates a cross-sectional view of a ground-penetrating portion of the non-rotating, ground-engaging tool shown in FIG. 2, particularly illustrating a soil sensor positioned within a cavity defined by the tool;

FIG. 4 illustrates a schematic view of one embodiment of a system for monitoring soil composition within a field in accordance with aspects of the present subject matter; and

FIG. 5 illustrates a flow diagram of one embodiment of a method for monitoring soil composition within a field in accordance with aspects of the present subject matter.

Repeat use of reference characters in the present specification and drawings is intended to represent the same or analogous features or elements of the present technology.

### DETAILED DESCRIPTION OF THE DRAWINGS

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

In general, the present subject matter is directed to systems and methods for monitoring soil composition within a field. Specifically, in several embodiments, as an agricultural machine is moved across a field, a controller of the disclosed system may be configured to receive data from a soil sensor installed or otherwise positioned within a cavity defined by a non-rotating ground-engaging tool (e.g., a tillage shank) of the machine. For example, in one embodiment, the cavity may include an opening defined by an aft surface of a ground-penetrating portion of the tool. The soil sensor may, in turn, be configured to emit an output signal(s) (e.g., an electromagnetic radiation signal(s)) through the opening for reflection off of the soil within the field. Moreover, the soil sensor may be configured to detect the reflected output signal(s) as a return signal(s), with one or more parameters (e.g., spectral parameter(s)) of the return signal(s) being indicative of the composition of the soil (e.g., the amount of organic matter, residue, and/or moisture within the soil). In this regard, the controller may be configured to determine the composition of the soil within the field across which the machine is being moved based on the received data. Thereafter, when the determined soil composition differs from a predetermined range of soil compositions, the controller may be configured to initiate one or more control actions associated with adjusting an operating parameter of the agricultural machine (e.g., the penetration depth(s) of and/or the down force being applied to the ground-engaging tool(s) of the machine).

The present subject matter will generally be described herein in the context of monitoring soil composition using a soil sensor positioned within a cavity defined by a ripper shank mounted on a tillage implement, such as a disc harrow. However, it should be appreciated that the disclosed system and method may also be used to monitor soil composition using a soil sensor positioned within a cavity defined by any other suitable non-rotating ground-engaging tool (e.g., a cultivator shank, sweep, tine, chisel, hoe, and/or the like) mounted on any other type of agricultural machine, such as another suitable type of implement (e.g., seeder, a planter, a fertilizer, and/or the like) and/or a suitable agricultural vehicle (e.g., tractor, a harvester, a self-propelled sprayer, and/or the like).

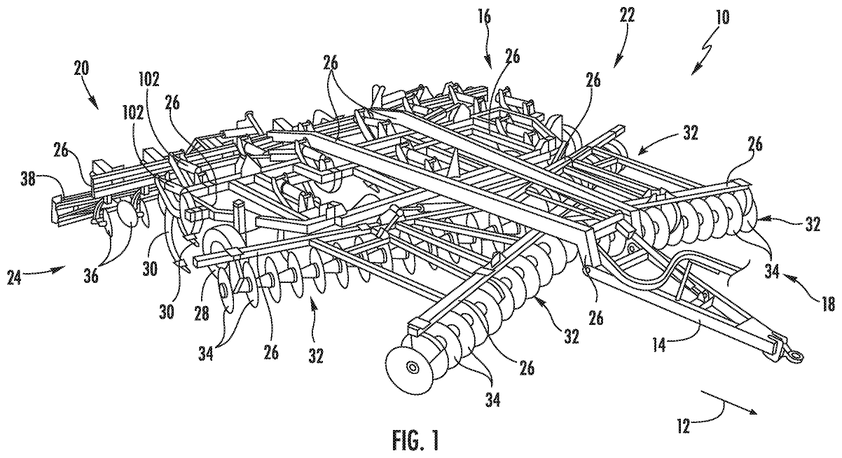

Referring now to the drawings. FIG. 1 illustrates a perspective view of one embodiment of an agricultural implement 10 in accordance with aspects of the present subject matter. As shown in the illustrated embodiment, the implement 10 may be configured to be towed across a field in a direction of travel (e.g., as indicated by arrow 12) by a work vehicle (not shown), such as a tractor or other agricultural work vehicle. The implement 10 may be coupled to the work vehicle via a hitch assembly 14 or using any other suitable attachment means.

The implement 10 may also include a frame 16. As shown, the frame 16 may extend longitudinally between a forward end 18 and an aft end 20. The frame 16 may also extend laterally between a first side 22 and a second side 24. In this respect, the frame 16 generally includes a plurality of structural frame members 26, such as beams, bars, and/or the like, configured to support or couple to a plurality of components. Additionally, a plurality of wheels 28 (one is shown) may be coupled to the frame 16 to facilitate towing the implement 10 in the direction of travel 12.

In several embodiments, the frame 16 may configured to support a plurality of shanks 30 configured to rip or otherwise till the soil as the implement 10 is towed across the field. In this regard, the shanks 30 may be configured to engage the soil as the tillage implement 10 is towed across the field. As will be described below, the shanks 30 may be configured to be pivotably mounted to the frame 16 to allow the shanks 30 to pivot out of the way of rocks or other impediments in the soil. As shown, the shanks 30 may be spaced apart from one another laterally between the first side 22 and the second side 24 of the frame 16. Furthermore, as will be described below, the implement 10 may include a plurality of biasing elements 102, with each biasing element 102 coupled between one of the shanks 30 and the frame 16. Although only two shanks 30 and biasing elements 102 are shown in FIG. 1, it should be appreciated that the implement 10 may generally include any number of shanks 30 and/or biasing elements 102 mounted on the frame 16.

In one embodiment, the frame 16 may be configured to support one or more gangs or sets 32 of disc blades 34. In general, each disc blade 34 may, for example, include both a concave side (not shown) and a convex side (not shown). Moreover, the various gangs 32 of disc blades 34 may be oriented at an angle relative to the travel direction 12 to promote more effective tilling of the soil. In the embodiment shown in FIG. 1, the implement 10 includes four gangs 32 of disc blades 34, with each gang 32 being coupled to the frame 16 longitudinally forward of the shanks 30. However, it should be appreciated that, in alternative embodiments, the implement 10 may include any other suitable number of disc gangs 34, such as more of fewer than four disc gangs 34. Furthermore, in one embodiment, the disc gangs 34 may be mounted longitudinally aft of the shanks 30, 32.

Additionally, as shown in FIG. 1, in one embodiment, the frame 16 may be configured to support other ground-engaging tools. For instance, in the illustrated embodiment, the frame 16 is configured to support a plurality of leveling blades 36 and rolling (or crumbler) basket assemblies 38. However, in other embodiments, any other suitable ground-engaging tools may be coupled to and supported by the frame 16, such as a plurality closing discs.

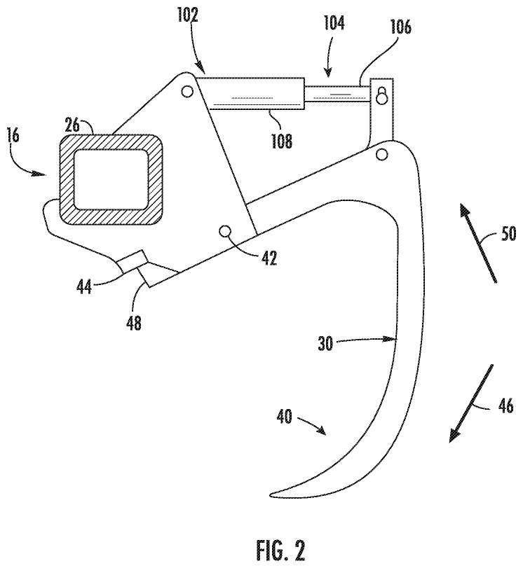

Referring now to FIG. 2, a side view of one embodiment of a shank 30 of the agricultural implement 10 is illustrated in accordance with aspects of the present subject matter. As indicated above, the shanks 30 may be configured to till or otherwise cultivate the soil. In this regard, one end of the shank 30 may include a ground-penetrating portion 40 configured to penetrate or otherwise engage the ground as the implement 10 is pulled across the field. The opposed end of the shank 30 may be pivotably coupled to the implement frame 16, such as at a pivot joint 42.

As shown in FIG. 2, a biasing element 102 may be coupled between the frame 16 and the shank 30. Specifically, in several embodiments, the biasing element 102 may be configured to bias the shank 30 to a predetermined shank position (e.g., a home or base position) relative to the frame 16. In general, the predetermined shank position may correspond to the shank position at which the shank 30 penetrates the soil to a desired depth. In one embodiment, the predetermined shank position for the shank 30 may be set by a corresponding mechanical stop 44. In operation, the biasing element 102 may permit relative movement between the shank 30 and the frame 16. For example, the biasing element 102 may be configured to bias the shank 30 to pivot relative to the frame 16 in a first pivot direction (e.g., as indicated by arrow 46 in FIG. 2) until its respective end 48 contacts the stop 44. The biasing element 102 may also allow the shank 30 to pivot away from the predetermined shank position (e.g., to a shallower depth of penetration), such as in a second pivot direction (e.g., as indicated by arrow 50 in FIG. 2) opposite the first pivot direction 46, when encountering rocks or other impediments in the field.

In several embodiments, the biasing element 102 may be configured as an actuator 104. In such embodiment, the actuator 104 may, in addition to biasing the shank 30 to the predetermined shank position, be configured to actively adjust the penetration depth of and/or the down force being applied to the shank 30. For example, in one embodiment, a first end of each actuator 104 (e.g., a rod 106 of each actuator 104) may be coupled to the shanks 30, while a second end of each actuator 104 (e.g., the cylinder 108 of each actuator 104) may be coupled to the frame 16. The rod 106 of the actuator 104 may be configured to extend and/or retract relative to the cylinder 108 to adjust the position of the shank 30 relative to the frame 16 in a manner that adjusts the penetration depth of and/or the downforce being applied to the shank 30. In one embodiment, the actuator 104 corresponds to a fluid-driven actuator, such as a hydraulic or pneumatic cylinder. However, in alternative embodiments, the actuator 104 may correspond to any other suitable type of actuator(s), such as an electric linear actuator(s). Additionally, in embodiments where the biasing element 102 is not configured to adjust the penetration depth of and/or the downforce being applied to the shank 30, the biasing element 102 may be configured as a suitable spring(s).

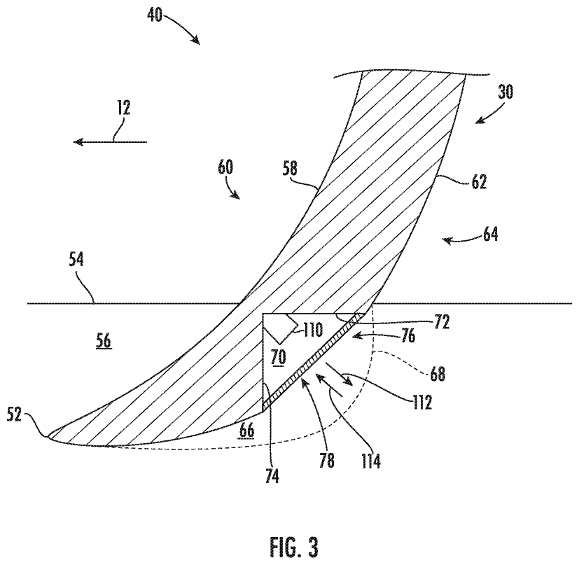

Referring now to FIG. 3, a cross-sectional view of the ground-penetrating portion 40 of the shank 30 shown in FIG. 2 is illustrated in accordance with aspects of the present subject matter. As indicated above, the ground-penetrating portion 40 of the shank 30 may generally be configured to penetrate the ground as the implement 10 is pulled across the field. As such, the shank 30 may include a tip 52 configured to pierce or otherwise penetrate a top surface 54 of the field. Furthermore, as shown, the shank 30 may include a leading surface 58 positioned on a forward side 60 of the shank 30 relative to the direction of travel 12. Similarly, the shank 30 may also include a trailing surface 62 positioned on an aft side 64 of the shank 30 relative to the direction of travel 12. Additionally, as shown in FIG. 3, the shank 30 may extend rearwardly and upwardly in a curved or arcuate manner from its tip 52 towards its non-ground-penetrating portion. However, in alternative embodiments, shank 30 may have any other suitable configuration, such as any other suitable shape.

When the implement 10 is moved across the field in the direction of travel 12, the ground-penetrating portion 40 of the shank 30 may be pulled through soil 56 such that the soil 56 flows around the ground-penetrating portion 40 in a manner that tills or otherwise works the soil 56. For example, when the shank 30 is pulled through the soil 56, the soil 56 may initially contact the leading surface 58 of the shank 30. A first portion of the soil 56 may flow around one side of the shank 30, while another portion of the soil 56 may flow around the opposed side of the shank 30. The portions of the soil around each side of the shank 30 may converge aft of the trailing surface 62 of the shank 30. In this regard, a void 66 in the soil 56 may be formed underneath and/or behind the ground-penetrating portion 40 of the shank 30 as the shank 30 is pulled through the soil 56. For instance, as shown in FIG. 3, the void 66 may be defined between the trailing surface 62 of the shank 30 and a location at which the portions of soil flowing around the shank 30 converge (e.g., as indicated by dashed line 68 in FIG. 3).

Furthermore, the shank 30 may define a cavity 70 therein. As will be described below, a soil sensor 110 may be installed or otherwise positioned within the cavity 70. The soil sensor 110 may, in turn, be configured to emit an output signal(s) for reflection off of the soil 56 and receive the reflected output signals as a return signal(s), with such return signals being indicative of the composition of the soil 54. Specifically, in several embodiments, the cavity 70 may be defined by the ground-penetrating portion 40 of the shank 30 such that, when the shank 30 is biased to its predetermined shank position, the cavity 70 is positioned beneath the soil surface 54. Furthermore, the cavity 70 may be positioned adjacent to the aft side 64 of the shank 30. For example, as shown in FIG. 3, the cavity 70 may be at least partially defined by a top surface 72 that extends forward in the direction of travel 12 from the trailing surface 62 toward the leading surface 58. In one embodiment, the top surface 72 may generally be parallel to the soil surface 54. Moreover, the cavity 70 may be at least partially defined by a forward surface 74 that extends upward from the trailing surface 62 toward the leading surface 58. As such, the top and forward surfaces 72, 74 may be oriented perpendicularly relative to each other such that the surfaces 72, 74 intersect at a generally right angle. In this regard, the cavity 70 may define a generally triangular cross-sectional shape. However, in alternative embodiments, the cavity 70 may have any other suitable configuration.

Additionally, the trailing surface 62 of the shank 30 may define an opening 76 of the cavity 70. In general, the opening 76 permits access to the cavity 70 such that the soil sensor 110 may be installed therein. Furthermore, as will be described below, the opening 76 may permit the output signal(s) emitted by the sensor 110 to exit the cavity 70 and reflected return signal(s) to enter the cavity 76. As shown in FIG. 3, the opening 76 may be positioned between the cavity 70 and the void 66 such that the output signal(s) are directed into the void 66.

In one embodiment, a covering or window 78 may be positioned within the opening 76 to prevent soil and/or moisture from entering the cavity 76 and potentially impacting the operation of the soil sensor 110. In this regard, the window 78 may correspond to any suitable device that may prevent soil/moisture from entering the cavity 76, while still allowing emitted output signal(s) to exit and reflected return signal(s) to enter the cavity 70. For example, in one embodiment, the window 78 may be a transparent or translucent component (e.g., a sheet/plate of polymeric material) that separates the cavity 70 from the void 66. Moreover, in the illustrated embodiment, the window 78 generally has a planar cross-section such that the output and/or return signals are not distorted thereby. However, in alternative embodiments, the cross-section of the window 78 may be curved (e.g., either in a concave or convex nature) such that the output and/or return signals are focused or dispersed. Furthermore, the window 78 may be any other suitable component that operates in a manner described above. In some embodiments, no window 78 may be positioned within the opening 76.

In accordance with aspects of the present subject matter, a soil sensor 110 may be installed or otherwise positioned within the cavity 70. In general, the soil sensor 110 may be configured to emit one or more output signals (e.g., as indicated by arrow 112 in FIG. 3) for reflection off of the soil 54. Specifically, as shown, the output signal(s) 112 emitted by the soil sensor 110 may travel through the cavity 70 and the opening/window 76/78 and into the void 66. Thereafter, the output signal(s) 112 may be reflected by the soil surface 68 defining the void 66 as a return signal(s) (e.g., as indicated by arrow 114 in FIG. 3) such that the return signal(s) travel through the void 66 and the opening/window 76/78 and into cavity 70. As such, the soil sensor 110 may be configured to receive the reflected return signal(s) 114. Furthermore, the soil sensor 110 may be mounted or positioned within the cavity 70 in any suitable manner that permits the sensor 110 to emit the output signal(s) 112 into the void 66 and receive the reflected return signal(s) 114. For example, in the illustrated embodiment, the soil sensor may be positioned within the cavity 70 at the intersection of the top and forward surfaces 72, 74 such that the output signal(s) 112 emitted by the sensor 110 are perpendicular to the opening/window 76/78. However, in alternative embodiments, the soil sensor 110 may be mounted on the top surface 72, the bottom surface 74, or at any other suitable surface or feature defining or within the cavity 70.

It should be appreciated that the soil sensor 110 may generally correspond to any suitable sensing device configured to function as described herein, such as by emitting one or more output signals for reflection off of the soil 54 and by receiving or sensing the return signal(s). For example, in one embodiment, the soil sensor 110 may include an emitter(s) configured to emit an electromagnetic radiation signal(s), such as an ultraviolet radiation signal(s), a near-infrared radiation signal(s), a mid-infrared radiation signal(s), or a visible light signal(s) for reflection off of the soil 54. The soil sensor 110 may also include a receiver(s) configured to receive the reflected electromagnetic radiation signal(s). One or more spectral parameter(s) (e.g., the amplitude, frequency, and/or the like) of the reflected electromagnetic radiation signal(s) may, in turn, be indicative of the composition of the soil 54. In this regard, the emitter(s) may be configured as a light-emitting diode (LED(s)) or other electromagnetic radiation-emitting device(s) and the receiver(s) may be configured as a photo resistor(s) or other electromagnetic radiation-receiving device(s). However, in alternative embodiments, the soil sensor 110 may have any other suitable configuration and/or components.

Moreover, it should be appreciated that installation of the soil sensor 110 within the cavity 70 defined by the shank 30 may provide one or more technical advantages. For instance, by positioning the soil sensor 110 within the cavity, the soil sensor 110 is able to capture data indicative of the composition of the soil 54 directly behind the shank 30 without affecting or otherwise interfering with the flow of soil around the shank 30. Furthermore, by positioning the cavity 70 within the shank 30 such that it is located underneath the soil surface 54, ambient light (e.g., sunlight) may not interfere with the output and/or return signals 112, 114.

Furthermore, it should be appreciated that the implement 10 may include one or more soil sensors 110. For example, in one embodiment, the implement 10 may only include one soil sensor 110. In such embodiment, only one shank 30 defines a cavity 70 in which a soil sensor 110 is installed. In another embodiment, the implement 10 may include a plurality of soil sensors 110. In such embodiment, several shanks 30 may each define a cavity 70 in which a soil sensor 110 is installed.

Additionally, it should be appreciated that the configuration of the implement 10 described above and shown in FIGS. 1-3 is provided only to place the present subject matter in an exemplary field of use. Thus, it should be appreciated that the present subject matter may be readily adaptable to any manner of machine configuration.

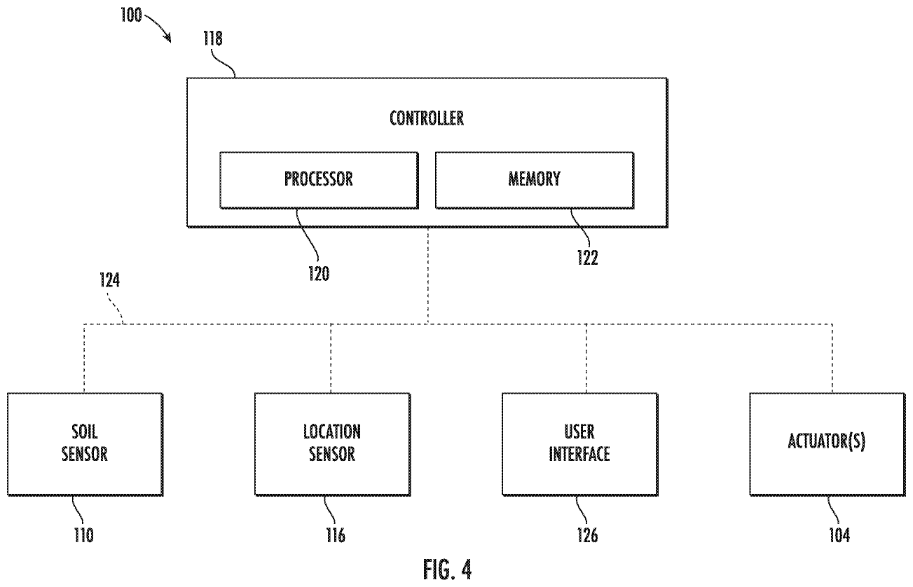

Referring now to FIG. 4, a perspective view of one embodiment of a system 100 for monitoring soil composition within a field is illustrated in accordance with aspects of the present subject matter. In general, the system 100 will be described herein with reference to the implement 10 described above with reference to FIGS. 1-3. However, it should be appreciated by those of ordinary skill in the art that the disclosed system 100 may generally be utilized with agricultural machines having any other suitable machine configuration.

As shown in FIG. 4, the system 100 may include a location sensor 116 provided in operative association with the implement 10 or an associated agricultural vehicle (not shown). In general, the location sensor 116 may be configured to determine the exact location of the implement 10 using a satellite navigation positioning system (e.g. a GPS system, a Galileo positioning system, the Global Navigation satellite system (GLONASS), the BeiDou Satellite Navigation and Positioning system, and/or the like). In such an embodiment, the location determined by the location sensor 116 may be transmitted to a controller(s) of the implement 10 and/or the associated vehicle (e.g., in the form coordinates) and stored within the controller's memory for subsequent processing and/or analysis. For instance, based on the known dimensional configuration and/or relative positioning between soil sensor 110 and the location sensor 116, the determined location from the location sensor 116 may be used to geo-locate the soil sensor 110 within the field.

In accordance with aspects of the present subject matter, the system 100 may include a controller 118 positioned on and/or within or otherwise associated with the implement 10 or an associated agricultural vehicle. In general, the controller 118 may comprise any suitable processor-based device known in the art, such as a computing device or any suitable combination of computing devices. Thus, in several embodiments, the controller 118 may include one or more processor(s) 120 and associated memory device(s) 122 configured to perform a variety of computer-implemented functions. As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 122 of the controller 118 may generally comprise memory element(s) including, but not limited to, a computer readable medium (e.g., random access memory (RAM)), a computer readable non-volatile medium (e.g., a flash memory), a floppy disc, a compact disc-read only memory (CD-ROM), a magneto-optical disc (MOD), a digital versatile disc (DVD), and/or other suitable memory elements. Such memory device(s) 122 may generally be configured to store suitable computer-readable instructions that, when implemented by the processor(s) 120, configure the controller 118 to perform various computer-implemented functions.

In addition, the controller 118 may also include various other suitable components, such as a communications circuit or module, a network interface, one or more input/output channels, a data/control bus and/or the like, to allow controller 118 to be communicatively coupled to any of the various other system components described herein (e.g., the actuator(s) 104, the soil sensor 110, and/or the location sensor 116). For instance, as shown in FIG. 4, a communicative link or interface 124 (e.g., a data bus) may be provided between the controller 118 and the components 104, 110, 116 to allow the controller 118 to communicate with such components 104, 110, 116 via any suitable communications protocol (e.g., CANBUS).

It should be appreciated that the controller 118 may correspond to an existing controller(s) of the implement 10 and/or an associated agricultural vehicle, itself, or the controller 118 may correspond to a separate processing device. For instance, in one embodiment, the controller 118 may form all or part of a separate plug-in module that may be installed in association with the implement 10 and/or the vehicle to allow for the disclosed systems to be implemented without requiring additional software to be uploaded onto existing control devices of the implement 10 and/or the vehicle. It should also be appreciated that the functions of the controller 118 may be performed by a single processor-based device or may be distributed across any number of processor-based devices, in which instance such devices may be considered to form part of the controller 118. For instance, the functions of the controller 118 may be distributed across multiple application-specific controllers, such as a navigation controller, an implement controller, and/or the like.

Furthermore, in one embodiment, the system 100 may also include a user interface 126. More specifically, the user interface 126 may be configured to provide feedback (e.g., feedback associated with composition of the soil 54) to the operator of the implement 10 and/or the associated agricultural vehicle. As such, the user interface 126 may include one or more feedback devices (not shown), such as display screens, speakers, warning lights, and/or the like, which are configured to provide feedback from the controller 118 to the operator. The user interface 126 may, in turn, be communicatively coupled to the controller 118 via the communicative link 124 to permit the feedback to be transmitted from the controller 118 to the user interface 126. In addition, some embodiments of the user interface 126 may include one or more input devices (not shown), such as touchscreens, keypads, touchpads, knobs, buttons, sliders, switches, mice, microphones, and/or the like, which are configured to receive user inputs from the operator.

In several embodiments, the controller 118 may be configured to determine the composition of the soil within the field across which the implement 10 is being moved. As described above, the implement 10 may include a soil sensor(s) 110 installed or otherwise positioned within a cavity 70 defined by one or more shank(s) 30. The soil sensor(s) 110 may be configured to emit the output signal(s) 112 through the corresponding opening(s) 76 and/or the window(s) 78 for reflection off of the soil within the field. Moreover, the soil sensor(s) 110 may be configured to detect the reflected output signal(s) as return signal(s) 114, with one or more parameters of the return signal(s) 114 being indicative of the composition of the soil. In this regard, the controller 118 may be configured to receive data from the soil sensor(s) 110 (e.g., via the communicative link 124) associated with the detected return signal(s) 114. Thereafter, the controller 118 may be configured to analyze/process the received data to determine the composition of the soil within the field. For instance, the controller 118 may include a look-up table(s), suitable mathematical formula, and/or algorithms stored within its memory 122 that correlates the received data to the soil composition of the field. In one embodiment, the controller 118 may be configured to store the determined soil composition of the field within its memory 122 and/or transmit the determined soil composition of the field to a remote device (e.g., a Smartphone, a tablet, a PC, a database server, and/or the like). Such soil composition data may, in turn, be used in planning and/or performing future agricultural operations.

It should be appreciated that the determined soil composition of the field may provide an indication of the amounts and/or concentrations of one or more constituents or components of the soil within the field. For example, in one embodiment, the determined soil composition may provide an indication of the amount and/or concentration of organic matter, residue, and/or moisture within the soil. However, in alternative embodiments, the determined soil composition may provide an indication any other suitable constituent or component of the soil.

Additionally, the controller 118 may be configured to generate a field map illustrating the soil composition at various locations within the field. More specifically, as described above, the controller 118 may be configured to geo-locate the position of the soil sensor(s) 110 within the field and determine the soil composition at the location(s) of the sensor(s) 110 as the implement 10 is being moved across the field. As such, the controller 118 may associate each soil composition determination with the position in the field where the determination was made. Thereafter, the controller 118 may be configured to generate a field map (e.g., a graphical field map) illustrating the soil composition at various positions within the field. For instance, the controller 118 may be configured to execute one or more algorithms stored within its memory 122 that generate the field map based on the data received soil sensor 110 and the location sensor 116 (e.g., via the communicative link 124). In one embodiment, the controller 118 may be configured to transmit instructions to the user interface 126 (e.g., the communicative link 124) instructing the user interface 126 to display the field map (e.g., a graphical field map).

Furthermore, the controller 118 may be configured to initiate one or more control actions when the determined soil composition differs from a predetermined range of soil compositions. Such control actions(s) may generally be associated with adjusting one or more operating parameters of the implement 10 in a manner that modifies the composition of the soil within the field. Specifically, in several embodiments, the controller 118 may be configured to compare the determined soil composition to the predetermined range of soil compositions. The predetermined range may, in turn, be indicative of a range of desired or acceptable soil compositions for the field, such as a desired or acceptable range(s) of the amount(s) or concentration(s) of one or more soil components/constituents within the field. Thereafter, when the determined soil composition differs from the predetermined range of soil compositions (thereby indicating that the soil within the field has too much or too little of a soil component(s)/constituent(s)), the controller 118 may be configured to adjust one or more operating parameters of the implement 10.

In one embodiment, the controller 118 may be configured to automatically adjust the penetration depth of and/or down force being applied to the ground-engaging tools (e.g., the shanks 30) of the implement 10 when the determined soil composition differs from the predetermined range of soil compositions. In such embodiment, the controller 118 may be configured transmit instructions to the actuator(s) 104 (e.g., via the communicative link 124) instructing the actuator(s) 104 to adjust the penetration depth(s) of and/or down force being applied to the associated shank(s) 30. However, in alternative embodiments, the controller 118 may be configured to adjust any other suitable operating parameter(s) of the implement 10, such as the penetration depth(s) of and/or down force(s) being applied to other ground-engaging tools (e.g., the disc blades 34) of the implement 10, the ground speed of the implement 10, and/or the like.

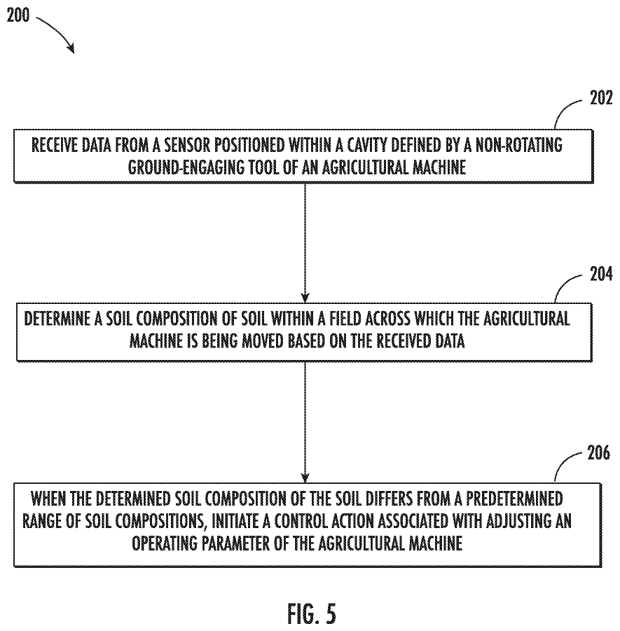

Referring now to FIG. 5, a flow diagram of one embodiment of a method 200 for monitoring soil composition within a field is illustrated in accordance with aspects of the present subject matter. In general, the method 200 will be described herein with reference to the agricultural implement 10 and the system 100 described above with reference to FIGS. 1-4. However, it should be appreciated by those of ordinary skill in the art that the disclosed method 200 may generally be implemented with any agricultural machine having any suitable machine configuration and/or any system having any suitable system configuration. In addition, although FIG. 5 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As shown in FIG. 5, at (202), the method 200 may include receiving, with a computing device, data from a sensor positioned within a cavity defined by a non-rotating ground-engaging tool of an agricultural machine. For instance, as described above, the controller 118 may be configured to receive data from a soil sensor 110 positioned within a cavity 70 defined by a shank 30 of an agricultural implement 10.

Additionally, at (204), the method 200 may include determining, with the computing device, a soil composition of soil with a field across which the agricultural machine is being moved based on the received data. For instance, as described above, the controller 118 may be configured to determine a soil composition of soil with a field across which the agricultural implement 10 is being moved based on the received data.

Moreover, as shown in FIG. 5, at (206), when the determined soil composition of the soil differs from a predetermined range of soil compositions, the method 200 may include initiating, with the computing device, a control action associated with adjusting an operating parameter of the agricultural machine. For instance, as described above, when the determined soil composition of the soil differs from a predetermined range of soil compositions, the controller 118 may be configured to one or more control action associated with adjusting an operating parameter of the agricultural implement. Such operating parameter(s) may include the penetration depth and/or the down force being applied to the shanks 30 of the implement 10.

It is to be understood that the steps of the method 200 are performed by the controller 118 upon loading and executing software code or instructions which are tangibly stored on a tangible computer readable medium, such as on a magnetic medium, e.g., a computer hard drive, an optical medium, e.g., an optical disc, solid-state memory, e.g., flash memory, or other storage media known in the art. Thus, any of the functionality performed by the controller 118 described herein, such as the method 20X), is implemented in software code or instructions which are tangibly stored on a tangible computer readable medium. The controller 118 loads the software code or instructions via a direct interface with the computer readable medium or via a wired and/or wireless network. Upon loading and executing such software code or instructions by the controller 118, the controller 118 may perform any of the functionality of the controller 118 described herein, including any steps of the method 200 described herein.

The term “software code” or “code” used herein refers to any instructions or set of instructions that influence the operation of a computer or controller. They may exist in a computer-executable form, such as machine code, which is the set of instructions and data directly executed by a computer's central processing unit or by a controller, a human-understandable form, such as source code, which may be compiled in order to be executed by a computer's central processing unit or by a controller, or an intermediate form, such as object code, which is produced by a compiler. As used herein, the term “software code” or “code” also includes any human-understandable computer instructions or set of instructions, e.g., a script, that may be executed on the fly with the aid of an interpreter executed by a computer's central processing unit or by a controller.

This written description uses examples to disclose the technology, including the best mode, and also to enable any person skilled in the art to practice the technology, including making and using any devices or systems and performing any incorporated methods. The patentable scope of the technology is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal language of the claims.

1. A system for monitoring soil composition within a field, the system comprising:
a non-rotating ground-engaging tool configured to be pulled through soil within the field in a manner that performs an agricultural operation on the field, the non-rotating ground-engaging tool defining a cavity therein, the cavity including an opening; and
a sensor positioned within the cavity, the sensor configured emit an output signal through the opening for reflection off of the soil within the field, the sensor further configured to detect the reflected output signal as a return signal,
wherein a parameter of the return signal is indicative of a soil composition of the soil within the field.

2. The system of claim 1, wherein the non-rotating ground-engaging tool corresponds to a tillage shank.

3. The system of claim 2, wherein the tillage shank comprises a forward side and an aft side, the window positioned adjacent to the aft side.

4. The system of claim 1, further comprising:
a window positioned within the opening.

5. The system of claim 1, wherein the output signal comprises an electromagnetic radiation signal.

6. The system of claim 5, wherein the electromagnetic radiation signal comprises at least one of an ultraviolet radiation signal, a near-infrared radiation signal, a mid-infrared radiation signal, or a visible light signal.

7. The system of claim 5, wherein the parameter of the return signal comprises a spectral parameter.

8. The system of claim 1, further comprising:
a controller communicatively coupled to the sensor, the controller configured to determine the soil composition of the soil based on data received from the sensor associated with the parameter of the return signal.

9. The system of claim 8, wherein the soil composition of the soil comprises at least one of an amount of organic matter within the soil, an amount of crop residue within the soil, or an amount of moisture within the soil.

10. The system of claim 8, wherein the controller is further configured to generate a field map identifying the soil composition of the soil at a plurality of locations within the field.

11. The system of claim 8, wherein the controller is further configured to compare the determined soil composition of the soil to a predetermined range of soil compositions.

12. The system of claim 11, wherein the controller is further configured to initiate an adjustment of a penetration depth of or a downforce being applied to the non-rotating ground-engaging tool when the soil determined composition differs from the predetermined range of soil compositions.

13. An agricultural implement, comprising:
a frame;
a non-rotating ground-engaging tool mounted on the frame, the non-rotating ground-engaging tool configured to be pulled through soil within the field in a manner that performs an agricultural operation on the field as the agricultural implement is moved across the field, the non-rotating ground-engaging tool defining a cavity therein, the cavity including an opening; and
a sensor positioned within the cavity, the sensor configured emit an output signal through the opening for reflection off of the soil within the field, the sensor further configured to detect the reflected output signal as a return signal,
wherein a parameter of the return signal is indicative of a soil composition of the soil within the field.

14. The agricultural implement of claim 13, wherein the non-rotating ground-engaging tool corresponds to a tillage shank.

15. The agricultural implement of claim 14, wherein the tillage shank comprises a forward side and an aft side, the window positioned adjacent to the aft side.

16. A method for monitoring soil composition within a field across which an agricultural machine is being moved, the agricultural machine including a non-rotating ground-engaging tool configured to be pulled through soil within the field in a manner that performs an agricultural operation on the field, the non-rotating ground-engaging tool defining a cavity therein, the cavity including an opening, the method comprising:
receiving, with a computing device, data from a sensor positioned within the cavity, the sensor configured emit an output signal through the opening for reflection off of the soil within the field, the sensor further configured to detect the reflected output signal as a return signal;
determining, with the computing device, a soil composition of the soil based on the received data; and
when the determined soil composition of the soil differs from a predetermined range of soil compositions, initiating, with the computing device, a control action associated with adjusting an operating parameter of the agricultural machine.

17. The method of claim 16, wherein the non-rotating ground-engaging tool corresponds to a tillage shank.

18. The method of claim 16, wherein the soil composition of the soil comprises at least one of an amount of organic matter within the soil, an amount of crop residue within the soil, or an amount of moisture within the soil.

19. The method of claim 16, further comprising:
generating, with the computing device, a field map identifying the soil composition of the soil at a plurality of locations within the field.

20. The method of claim 16, wherein the control action comprises adjusting a penetration depth of or a downforce being applied to the non-rotating ground-engaging tool.
